(19) United States
(12) Patent Application Publication
(10) Pub. No.: US 2004/0243044 A1
(43) Pub. Date: Dec. 2, 2004

(54) Hemostatic wound dressing

(76) Inventors: Stephen A. Penegor, Watertown, MN (US); Howard Root, Excelsior, MN (US); James A. Murto, Maple Grove, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 10/452,821

(22) Filed: Jun. 2, 2003

(51) Int. Cl. A61F 13/00

(52) U.S. Cl. 602/48

(57) ABSTRACT

A hemostatic dressing for applying a hemostatic material such as lyophilized thrombin to a wound. The wound dressing includes an impermeable backing material and a sealing member to provide a seal against the skin. The wound dressing enables the application of pressure in order to seal the sealing member against the patient's skin to the wound without the need for attendance by a health care professional. The seal may be applied by a compressive band or by application of a pressure sensitive adhesive. The wound dressing creates a sealed localized procoagulation environment to facilitate the formation of a clot initiated by the presence of substantial quantities of externally supplied lyophilized thrombin or another dry coagulant.

### FIELD OF THE INVENTION

The present invention relates to a hemostatic wound dressing.

### BACKGROUND OF THE INVENTION

Many medical procedures in use today involve the insertion of a medical device into a living body in order to pierce the wall of a blood vessel or other similar structures within the body. Instruments such as biopsy needles, laparascopes, trochars, introducers and various other probes may be inserted into the body. The surgical insertion of such instruments creates a wound which, of course, tends to bleed. For example, patients undergoing periodic kidney dialysis may incur arteriopunctures several times weekly.

In order to stop the bleeding of such puncture wounds, it is common for medical personal to apply pressure to the wound for a substantial period of time to facilitate hemostasis. Direct pressure is often applied digitally, that is, with the fingers. The need to apply such pressure for extended periods of time leads to a less than efficient utilization of highly skilled medical personnel. Of particular concern are situations that require that digital pressure be applied for longer than five minutes to achieve a 95% success rate of stopping bleeding. Such needs arise particularly acutely in the case of arteriopunctures of major arteries.

A variety of pressure dressings have been utilized in an effort to provide appropriate pressure to create hemostasis. Pressure dressings stop blood flow mechanically and are generally a thick gauze, cotton or other absorbent breathable material in pad form applied to the wound.

Another approach to creating hemostasis is to apply an agent that accelerates blood clotting in the wound. The clot stops hemorrhaging by physically blocking the outflow of blood and provides a supporting structure for the formation of scar tissue and thus begins the healing process. One such clotting agent is, thrombin, a naturally produced protein agent, is available in either a liquid solution or a lyophilized (freeze-dried) form. During coagulation thrombin cleaves fibrinogen in the blood to form fibrin, leading to the ultimate step in coagulation and the formation of a fibrin clot.

In U.S. Pat. No. 6,447,799, B1 issued to Ullman, a hemostatic kit is disclosed that includes an absorbent pad, a thrombin retainer and a non-stick backing. The thrombin retainer holds a measured quantity of thrombin in liquid solution. The thrombin retainer can be broken to release the thrombin solution which then soaks the absorbent pad. The thrombin soaked gelatin foam pad of this invention can then be applied to a wound with pressure in order to stem bleeding.

Other approaches to applying medications to body sites include those disclosed in U.S. Pat. No. 4,899,739 issued to Konishi, and U.S. Pat. No. 3,580,254 issued to Stewart. These patents both disclose devices for releasing medication from a sealed storage receptacle into a bandage-like dressing utilizing permeable backing.

While these approaches represent a possible solution to the problems of dressings for wounds such as surgical insertions that require more than five minutes of digital pressure to stop bleeding, each of these approaches allow blood from the wound to seep from the wound area without being coagulated. As a result, these devices are not entirely effective in stemming bleeding from these kinds of significant wounds.

It would be beneficial to have a device for applying a hemostatic agent to a wound, with pressure, without the need for constant attention from a health professional during the time of application that could overcome the limitations of existing techniques.

### SUMMARY OF THE INVENTION

The present invention is a hemostatic wound dressing that prevents leakage of blood from the region occupied by the hemostatic material and the area immediately adjacent to the wound. The present invention facilitates hemostasis following percutaneous and transcutaneous procedures. The invention further allows the application of pressure to the wound without the need for constant attendance from a health care professional.

The invention generally includes a hemostatic dressing for providing a hemostatic material, such as lyophilized thrombin, in a ready-to-use form. The invention further includes an impermeable backing material and a sealing member to create a seal with the skin. One embodiment of the present invention provides for the application of pressure in order to seal the sealing member against the patient's skin surrounding the wound without the need for attendance by a health care professional. The pressure may be applied by a compressive band or by an adhesive bandage-like application. The pressure application utilized with the invention is not intended to, or sufficient to stop bleeding by mechanical or hydraulic pressure or force. Rather, the application of pressure by this arrangement creates a sealed localized procoagulation environment in order to facilitate the formation of a clot initiated by the presence of substantial quantities of externally supplied lyophilized thrombin or similar preformed coagulant. Preferably, the localized procoagulation environment facilitates and the exclusion of ambient air.

A first embodiment of the invention includes a gasket supported by two arms of unequal length. The ends of the unequal arms are operably, slidably connected with a circumferential band adapted to encircle a body part in order to hold the hemostatic dressing in place. The circumferential band is not a pressure applying band in the conventional medical sense. It does not serve to stop bleeding by application of direct pressure to a wound. Rather, this embodiment of the invention provides a localized procoagulant environment that is maintained with a gasket and an impermeable backing. Blood vessels in the area of the wound are not occluded by operation of the circumferential band due to the arms extended from the gasket. In other words, a tourniquet like situation is undesirable and avoided.

A second embodiment of the invention includes an impermeable pressure sensitive adhesive backing which also seals around the perimeter of the procoagulant material to create a localized environment for retaining blood to effectively interacts with the procoagulant material. This embodiment of the invention also provides a localized, procoagulant environment that is preferably anaerobic. In this embodiment, the seal minimizes air ingress and prevents fluid egress to the environment occupied by the procoagulant material through an adhesive bond between the impermeable backing and the skin of the patient rather than a pressure operated gasket.

A third embodiment includes a rigid gasket disk to seal around the preformed coagulant and a flexible pressure sensitive adhesive support for securing the disk-coagulant assembly to the skin.

### BRIEF DESCRIPTION OF THE DRAWINGS

FIG. 1 is a partially exploded, perspective view of a first embodiment in accordance with the present invention;

FIG. 2 is a front plan view of the embodiment of FIG. 1;

FIG. 3 is a perspective view of the embodiment of FIG. 1 from another angle;

FIG. 4 is a top plan view of the embodiment of FIG. 1;

FIG. 5 is a perspective view of a backplate in accordance with the present invention;

FIG. 6 is a top plan view of a second embodiment of the present invention;

FIG. 7 is a top plan view of the second embodiment of the invention including a partially opened removable backing;

FIG. 8 is an exploded elevational view of the second embodiment of the present invention;

FIG. 9 is a perspective view of a third embodiment in accordance with the present invention;

FIG. 10 is a perspective view of the embodiment of FIG. 8 from a reverse angle; and

FIG. 11 is a perspective view of a support block in accordance with an embodiment of the present invention.

### DETAILED DESCRIPTION OF THE PREFERRED EMBODIMENTS

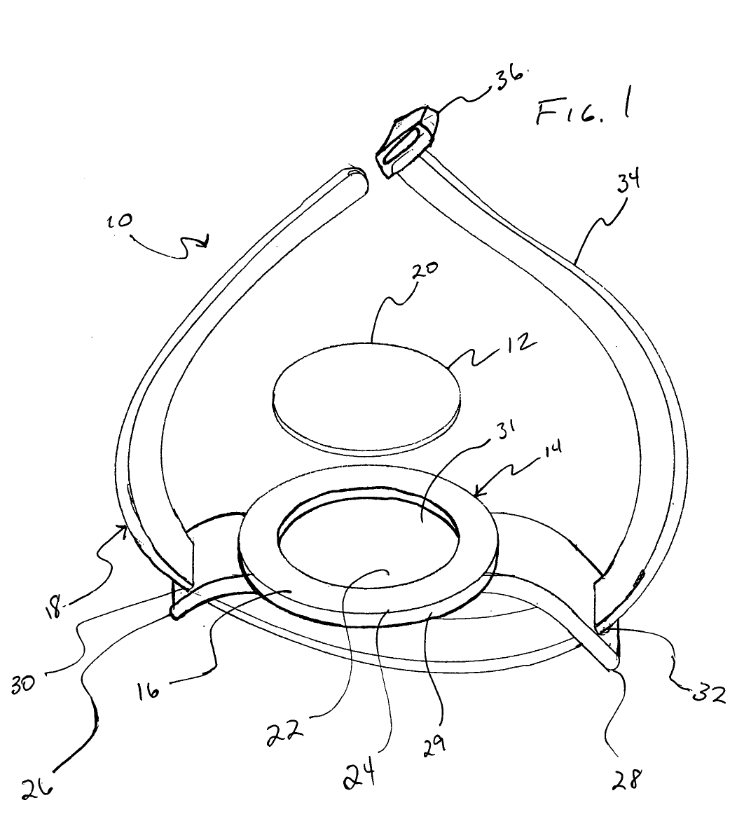

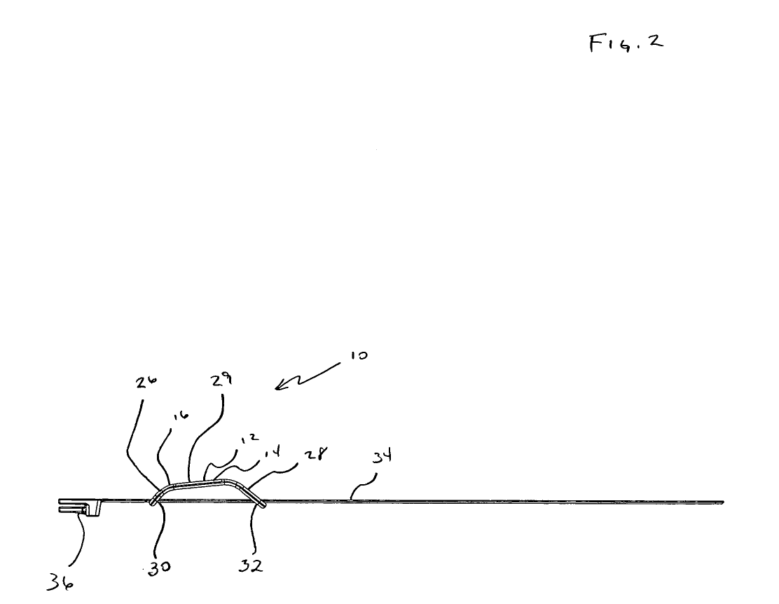

Referring to FIGS. 1, 2 and 3, a first embodiment of the hemostatic dressing 10 generally includes preformed procoagulant 12, impermeable backing 14, sealing member 16, and securement 18. Referring particularly to FIG. 1, procoagulant 12 preferably includes lyophilized thrombin. Procoagulant 12 is preferably in the form of a disk or pad having a thickness of between 1.0 and 10.0 millimeters. The disk may have a diameter of one to two centimeters. Desirably, procoagulant 12 contains at least ten units of thrombin or an amount of another procoagulant having similar level of procoagulant activity.

The shape of procoagulant 12 may be circular or polygonal, and preferably conforms to a shape of a perimeter 20 of the sealing member 16. Procoagulant 12 is supported by and/or adhered to impermeable backing 14 and surrounded at perimeter 20 by sealing member 16.

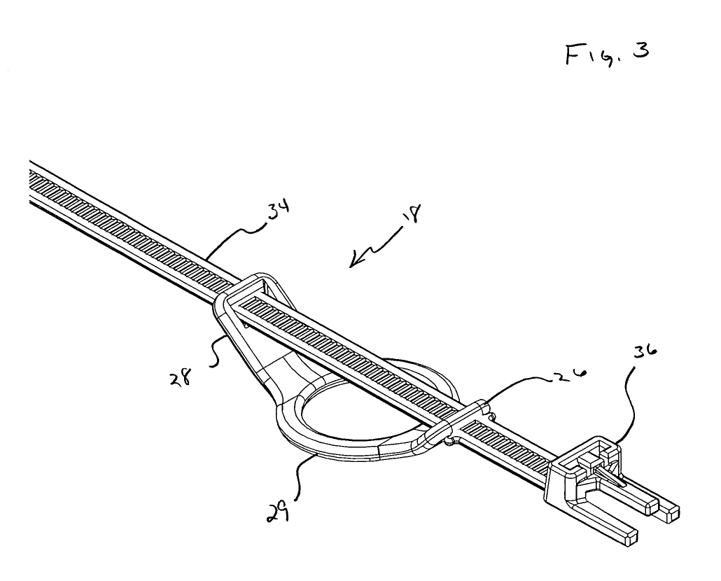

Still referring to FIGS. 1, 2 and 3, in this embodiment of the invention impermeable backing 14 generally includes back plate 22, annulus 24, short arm 26, and long arm 28. Referring to FIG. 3, back plate 22 preferably is a flat disc shaped structure having a depression 29 therein formed from a material that is impermeable to ambient air and to the components of procoagulant 12.

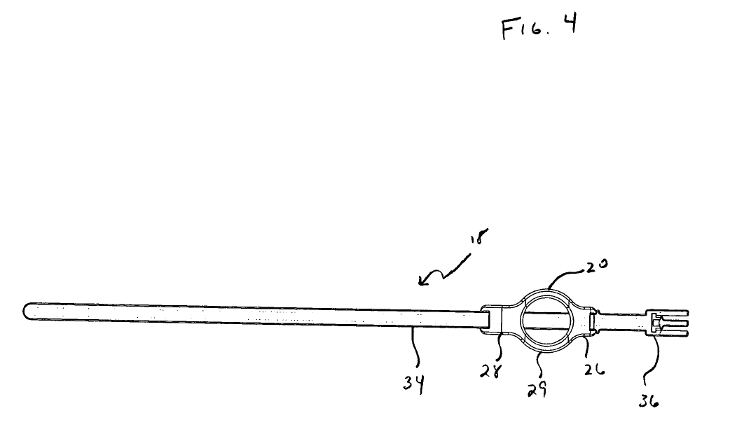

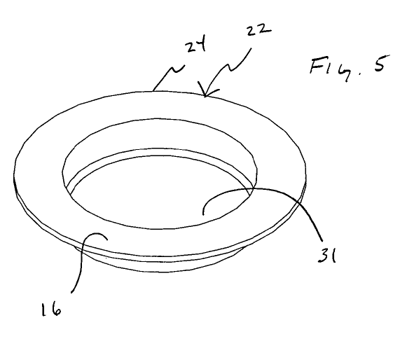

Referring to FIGS. 3 and 4, back plate 22 is joined to annulus 24 and supported thereby. Back plate 22 and annulus 24 cooperate in concert with the skin of the patient in the vicinity of the wound, to create an enclosed procoagulant microenvironment. Backplate 22 and annulus 24 are depicted in FIG. 5 as generally circular but can be any shape desired. Short arm 26 and long arm 28 may be formed as an interconnected unit joined by ring 29, as depicted in FIG. 3. Back plate 22 and annulus 24 may be formed as an integral unit and joined to ring 29 in a press fit fashion.

Short arm 26 is operably joined to back plate 22 and annulus 24. Short arm 26 is angled or curved away from procoagulant 12. Short arm 26 is pierced by slot 30. Slot 30 is sized and proportioned to accommodate securement 18.

Long arm 28 is operably connected to back plate 22 and annulus 24. Long arm 28 is longer in length than short arm 26 and is pierced by slot 32. Slot 32 is sized and proportioned to allow passage of securement 18 therethrough.

In this embodiment, sealing member 16 may be integrally formed as part of annulus 24 or may be a separate operably connected structure. Sealing member 16 is preferably raised relative to procoagulant 12 and is preferably formed from a biocompatible and hypoallergenic material. Sealing member 16 is structured to provide a leak resistant seal between sealing member 16 and the skin of a patient.

Referring to FIGS. 1 and 3 in this first embodiment of the invention, securement 18 includes circumferential band 34 and clasp 35. Circumferential band 34 is sized and proportioned in order to pass through slot 30 and slot 32. Circumferential band 34 is further sized and proportioned to encompass a desired body part. For example, if used to encourage hemostatis in a puncture wound used to access the femoral artery, circumferential band 34 should be proportioned to encompass the circumference of the upper thigh region of a human being.

Circumferential band 34 may also support a comfort pad (not shown). Comfort pad may be a gauze pad or another material to cushion the skin under circumferential band 34. For example, a foam or fabric pad would also be appropriate.

Clasp 35 may include a ratchet mechanism 36. Circumferential band 34 may include a plurality of closely spaced ratchet positions 37, as best seen in FIG. 3, so that when circumferential band 34 interacts with clasp 35 the circumference encircled by circumferential band 34 may be adjusted essentially infinitely. Circumferential band 34 and clasp 35 may interact in any way known in the art in order that the circumference encompassed by circumferential band 34 when secured by clasp 35 is variable within a range required to encompass a desired body part. Clasp 35 may be releasable in order to facilitate adjustment and/or removal of the circumferential band 34 relative to a body part. Any other length adjusting mechanism and/or bond retention mechanism known may also be utilized to make circumferential band 34 length adjustable.

Procoagulant 12 is preferably secured by an adhesive or mechanical means to impermeable backing 14 at back plate 22. Procoagulant 12 may be placed into depression 31. Procoagulant 12 may also, be infused, absorbed or otherwise integrated into impermeable backing 14.

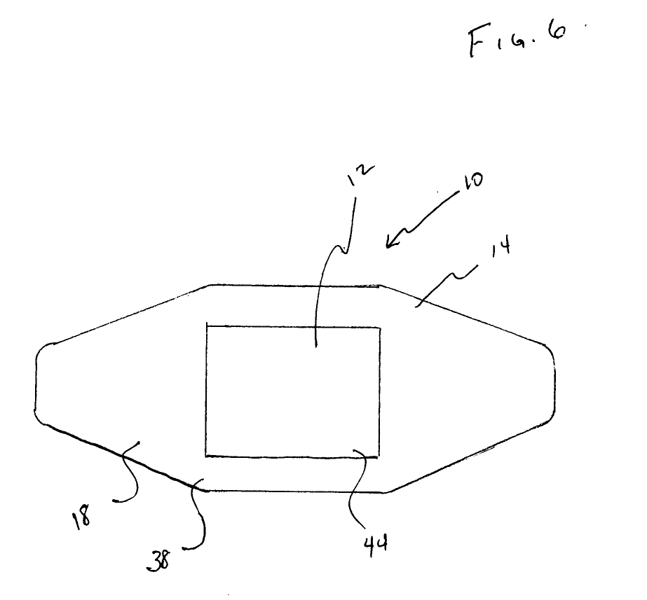

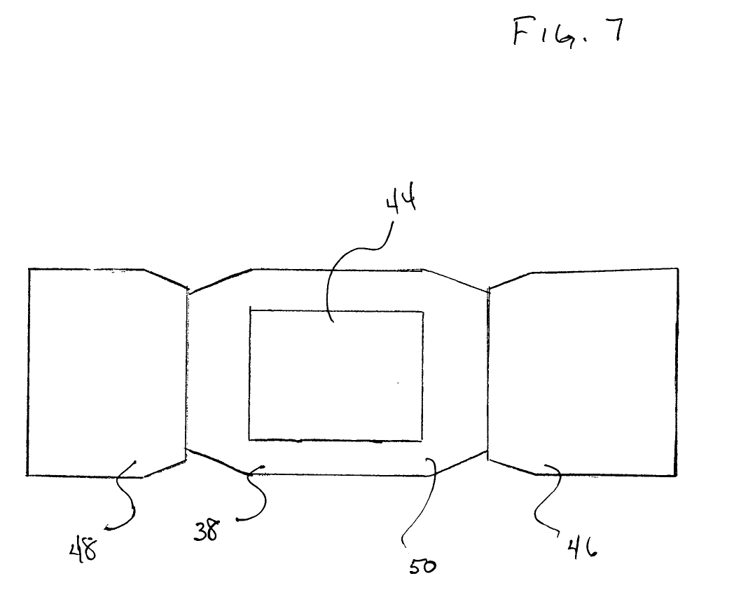

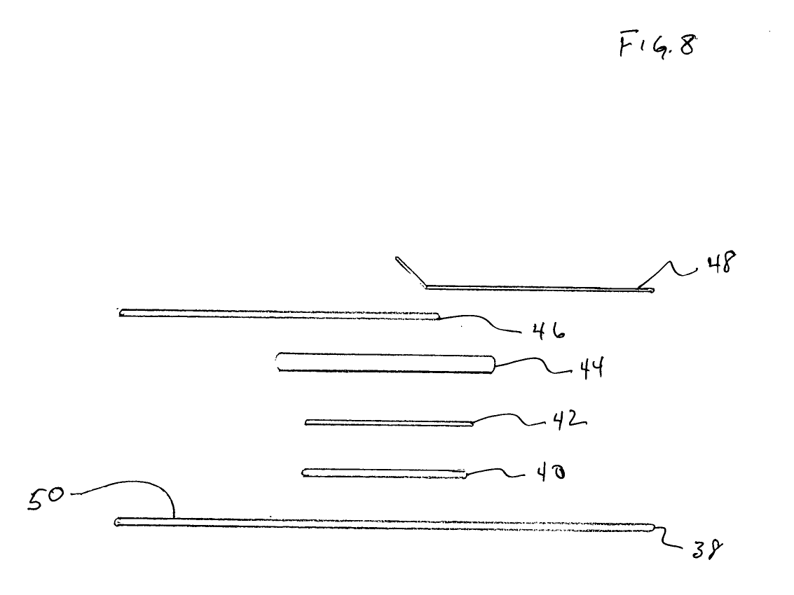

FIGS. 6-8 depict a second embodiment of the invention. The second embodiment of hemostatic dressing 10 also includes procoagulant 12, impermeable backing 14, and securement 18. In this embodiment, impermeable backing 14 includes flexible impermeable membrane 38 that also acts as the securement 18. Flexible impermeable membrane 38 supports absorbent pad 40, nonstick pad 42, quadrilateral procoagulant 44, first peelable layer 46, and second peelable layer 48.

Flexible impermeable membrane 38 is substantially impermeable to air. Flexible impermeable membrane 38 is coated on a first side thereof with pressure sensitive adhesive 50. Preferably, pressure sensitive adhesive 50 secures adsorbent pad 40 to flexible impermeable membrane 38. Pressure sensitive adhesive 50 also removably secures flexible impermeable membrane 38 to first peelable layer 46 and second peelable layer 48. First peelable layer 46 and second peelable layer 48 are formed of a material that will adhere to pressure sensitive adhesive 50 in a releasable fashion so that first peelable layer 46 and second peelable layer 48 can be removed from flexible impermeable membrane 38 readily to prepare hemostatic dressing 10 for use. Pressure sensitive adhesive 50 is preferably substantially impermeable to ambient air and forms an adhesive seal that minimizes any exchange of ambient air from the anaerobic hemostatic region and prevents egress of bodily fluids.

Absorbent pad 40 is covered by nonstick pad 42. Nonstick pad 42 preferably has the quality of being non-adherent to wounds and surrounding skin but allows wound exudates such as serum, blood or liquid to pass therethrough so as to be absorbed by absorbent pad 40. Quadrilateral procoagulant 44 covers nonstick pad 42 and is further covered by first peelable layer 46 and second peelable layer 48. Procoagulant 12 can be infused, or otherwise integrated into absorbent pad 40 or nonstick pad 42 as well.

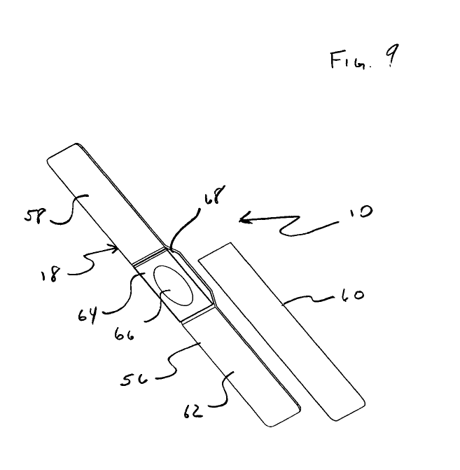

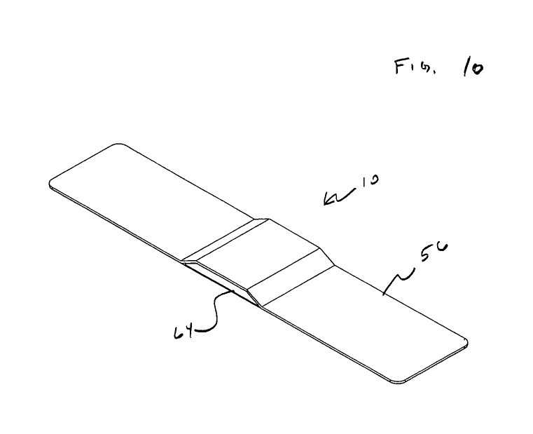

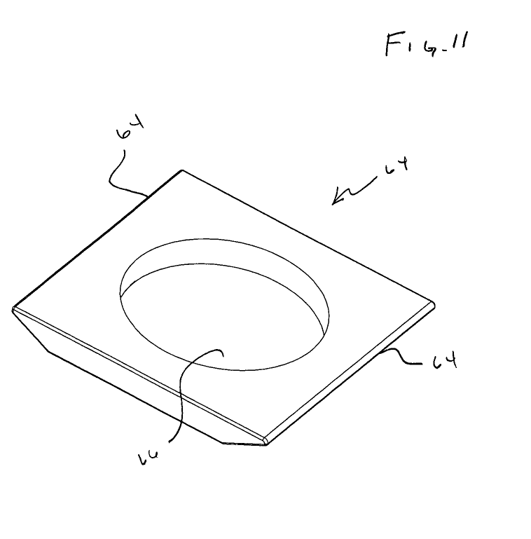

FIGS. 9-11 depict a third embodiment of the invention. This embodiment generally includes preformed procoagulant 12, impermeable backing 14, sealing member 16, and securement 18.

Impermeable backing 14 generally includes back plate 22 and annulus 24 which are similar in structure to those in the first embodiment disclosed above and depicted in FIG. 5. Sealing member 16 may be integrally formed as part of annulus 24 or may be a separate operably connected structure. Sealing member 16 is preferably raised relative to procoagulant 12 and is preferably formed from a biocompatible and hypoallergenic material. Sealing member 16 is structured to provide a leak resistant seal between sealing member 16 and the skin of a patient. Annulus 24 is desirably rigid enough to resist blood pressure in a large artery but flexible enough to seal with the skin prevent leakage of blood from the localized procoagulant environment.

In this embodiment, securement 18 includes a flexible self adhesive membrane 56. Flexible self adhesive membrane 56 is temporarily join to first peelable layer 58 and second peelable layer 60 by pressure sensitive adhesive 62. Pressure sensitive adhesive 62 is also adapted to provide a secure adhesion to the skin of the treated patient.

Securement 18 also preferably includes a support block 64. Support block 64 is preferably generally rectangular in shape and is pierced by back plate aperture 66. Backplate aperture 66 is sized and shaped to accept back plate 52, desirably, in a press fit fashion. Backplate aperture 66 and backplate 52 may be of any shape desired.

Support block 64 also defines beveled edge 68. Support block 64 is joined to flexible adhesive membrane 56. The joining may be accomplished by pressure sensitive adhesive 62 or by any other means known to the art.

In operation, the invention is utilized following a medical procedure that creates a skin wound. For example, after completion of kidney dialysis and removal of a dialysis needle, hemostatic dressing 10 is placed in contact with the patient's skin so that procoagulant 12 is in proximate contact with the wound and the nearby surrounding area. Thus, procoagulant 12 is enclosed and contained between impermeable backing 14 and the patient's skin. Sealing member 16 encircles procoagulant 12 and prevents procoagulant 12 leakage from the localized area.

The health care provider then grasps circumferential band 34 and manipulates it to encompass the limb or body part to which the hemostatic dressing 10 is to be secured. Once circumferential band 34 encompasses the body part, clasp 35 is utilized to secure circumferential band 34 about that body part. Clasp 35 and circumferential band 34 are adjusted so that procoagulant 12 is positioned over the wound and so that sealing member 16 is sealed against the patient's skin. Circumferential band 34 is tightened only enough to provide a tight seal between sealing member 16 and the patient's skin. It is noted that the interaction of circumferential band 34 with short arm 26 and long arm 28 allows sufficient pressure to be applied to sealing member 16 to form a tight seal without applying enough pressure to occlude arteries or veins beneath the skin. The structure of this embodiment prevents a tourniquet effect from occurring.

Once hemostatic dressing 10 is in place and secured, procoagulant 12 interacts with blood and wound exudate to be released directly into the wound and surrounding vicinity in order to facilitate coagulation and ultimate healing of the wound. Wound exudates dissolve and rehydrate thrombin from procoagulant 12 and the thrombin then interacts with the wound exudates within the localized procoagulant environment to initiate or speed the clotting process.

Referring to FIGS. 6, 7, and 8, when utilizing another embodiment of the invention, the physician removes first peelable layer 46 and second peelable layer 48 from flexible impermeable membrane 38. Doing so exposes pressure sensitive adhesive 50. The physician applies hemostatic dressing 10 so that quadrilateral procoagulant 44 is positioned over the wound in the skin. The physician then applies pressure to the non-adhesive side of flexible impermeable membrane 38 so that pressure sensitive adhesive 50 adheres to the skin of the patient on either side of the wound as well as around the perimeter of quadrilateral procoagulant 44. This produces a locally contained anaerobic procoagulant environment in the vicinity of the wound to facilitate the clotting and healing process.

Referring to FIG. 5, this embodiment of the invention is used in a fashion generally similar to the embodiment that is depicted in FIGS. 6, 7 and 8. Care should be taken to apply flexible self adhesive membrane 56 so that pressure sensitive adhesive 62 holds annulus 54 firmly against the skin of the patient thus creating a tight seal.

The present invention may be embodied in other specific forms without departing from the central attributes thereof, therefore, the illustrated embodiments should be considered in all respects as illustrative and not restrictive, reference being made to the appended claims rather than the foregoing description to indicate the scope of the invention.

What is claimed:

1. A hemostatic wound dressing for wounds that are exuding fluid, comprising:
a preformed coagulant having at least a first surface and a second surface;
structure retaining the preformed coagulant in position relative to a wound on a patient, such that the first surface of the coagulant is exposable to the wound and the structure defines a seal substantially around the wound; and
a covering operably attached to the structure and extending over the second surface, the covering and the structure defining a volume that substantially retains the preformed coagulant proximate the wound as fluid is exuded by the wound.

2. The wound dressing of claim 1, wherein the preformed coagulant is selected from the set consisting of a dry coagulant, a solid coagulant, a semisolid coagulant, a lyophilized coagulant and any combination thereof.

3. The wound dressing of claim 1, wherein the preformed coagulant is lyophilized thrombin.

4. The wound dressing of claim 1, wherein the structure and the covering are integrally formed of an adhesive strip of flexible material operably attached to the second surface of the preformed coagulant and extending beyond a periphery of the second surface of the preformed coagulant.

5. The wound dressing of claim 1, wherein the structure is an adhesive strip of flexible material extending beyond a periphery of the second surface of the preformed coagulant and the covering is a separate layer of material.

6. The wound dressing of claim 5, wherein the adhesive strip of flexible material covers the preformed coagulant and the separate layer of material is interposed between the preformed coagulant and the adhesive strip of flexible material.

7. The wound dressing of claim 1, wherein the wound is a significant wound for which digital pressure of at least five minutes is required to achieve a ninety five percent success rate of stopping bleeding.

8. The wound dressing of claim 1, wherein the structure comprises:
a retaining structure defining a cavity in which the preformed coagulant is retained, the cavity having a perimeter having a surface defining the seal surrounding the wound; and
a band operably connected to the retaining structure to maintain a position of the retaining structure relative to the wound.

9. The wound dressing of claim 8, wherein the retaining structure includes a pair of arms extending at least partially upward from the wound, each arm having attachment structure to operably attach the arm to the band such that the interaction of the arms and the band prevent a tourniquet effect from reducing blood flow.

10. The wound dressing of claim 9, wherein the attachment structure defines an aperture through which the band passes.

11. The wound dressing of claim 9, wherein the pair of arms comprises two arms of unequal length.

12. The wound dressing of claim 8, wherein the band has a pair of end portions and further comprises a clasp mechanism to secure the pair of end portions together.

13. The wound dressing of claim 12, wherein the clasp mechanism operates to releasably secure the pair of end portions together.

14. The wound dressing of claim 1, wherein the structure comprises:
a retaining structure defining a cavity in which the preformed coagulant is retained, the cavity having a perimeter having a surface defining the seal surrounding the wound; and
a self adhesive membrane operably connected to the retaining structure to adhere to a skin of the patient to maintain a position of the retaining structure relative to the wound.

15. A hemostatic wound dressing, comprising:
preformed means for enhancing coagulation of blood;
means for retaining the coagulation means in contact with a wound and surrounding skin; and
means for sealing the means for retaining to the skin of a patient so as to create a locally contained procoagulant environment that maintains the coagulation means within the procoagulant environment as fluid is exuded by the wound.

16. The wound dressing as claimed in claim 15, in which the means for enhancing coagulation is selected from a group consisting of a dry coagulant, a solid coagulant, a semisolid coagulant, a lyophilized coagulant and any combination thereof.

17. The wound dressing as claimed in claim 15, in which the means for enhancing coagulation comprises lypholized thrombin.

18. The wound dressing as claimed in claim 15, in which the means for retaining is integrally formed of an adhesive strip of flexible material operably attached to the means for enhancing coagulation and extending beyond a periphery of the means for enhancing coagulation.

19. The wound dressing as claimed in claim 15, in which the means for retaining is an adhesive strip of flexible material extending beyond a periphery of the means for enhancing coagulation and the means for sealing includes a separate layer of material.

20. The wound dressing as claimed in claim 19, in which the adhesive strip of flexible material covers the means for enhancing coagulation and the separate layer of material is interposed between the means for enhancing coagulation and the adhesive strip of flexible material.

21. The wound dressing as claimed in claim 15, in which the wound is a significant wound for which digital pressure of at least five minutes is required to achieve a ninety five percent success rate of stopping bleeding.

22. The wound dressing as claimed in claim 15, in which the means for retaining comprises:
a retaining structure enclosing the means for enhancing coagulation in a depression, the retaining structure having a perimeter for sealing around the wound; and
a band operably connected to the retaining structure to maintain the retaining structure in a desired position relative to the wound.

23. The wound dressing as claimed in claim 22, in which the retaining structure includes a pair of arms extending at least partially upward from the wound, each arm having attachment structure to operably attach the arm to the band such that the interaction of the arms and the band prevent a tourniquet effect from reducing blood flow.

24. The wound dressing of claim 23, in which the attachment structure defines an aperture through which the band passes.

25. The wound dressing of claim 23, in which the pair of arms comprises two arms of unequal length.

26. The wound dressing of claim 22, wherein the band has a pair of end portions and further comprises a clasp mechanism to secure the pair of end portions together.

27. The wound dressing of claim 26, wherein the clasp mechanism operates to releasably secure the pair of end portions together.

28. The wound dressing of claim 15, in which the means for retaining comprises:
a retaining structure enclosing the means for enhancing coagulation in a depression, the retaining structure having a perimeter for sealing around the wound; and
a self adhesive membrane to adhere to a skin of the patient, the self adhesive membrane being operably connected to the retaining structure to maintain the retaining structure in a position relative to the wound.

29. A method of manufacturing a wound dressing for dressing a wound in a skin of a patient comprising the steps of:
supplying an aliquot of preformed coagulant;
enclosing the aliquot in a space in a retaining structure so that the aliquot of preformed coagulant is enclosed on all sides but one the one side being encircled by a seal structure, the seal structure being adapted to seal with the skin; and
providing and arranging a securing structure operably connectable with the retaining structure and adapted to sealingly apply the seal structure and the retaining structure to the skin so that when applied the performed coagulant is surrounded by the retaining structure and the skin to create a localized procoagulant environment.

30. The method as claimed in claim 29, further comprising the step of selecting the preformed coagulant from a set consisting of a dry coagulant, a solid coagulant, a semisolid coagulant, a lyophilized coagulant and any combination thereof.

31. The method as claimed in claim 29, further comprising the step of selecting lypholized thrombin as the preformed coagulant.

32. The method as claimed in claim 29, further comprising the step of selecting securing structure from a set consisting of a pressure sensitive adhesive membrane, a band and clasp assembly and an adjustable band and clasp assembly.

33. The method as claimed in claim 29, further comprising the step of selecting the sealing structure from a group consisting of a gasket and an adhesive seal.

34. The method as claimed in claim 29, further comprising the steps of:
utilizing a retaining structure defining a cavity in which the preformed coagulant is enclosed, the cavity having a perimeter having a surface defining the seal structure surrounding the wound; and
in which the securing structure comprises a band operably connected to the retaining structure.

35. The method as claimed in claim 29, further comprising the step of operably connecting the securing structure to the retaining structure with a pair of arms extending at least partially upward from the retaining structure.

36. The method as claimed in claim 34, further comprising the step of operably connecting the securing structure to the retaining structure with a pair of unequal length arms extending at least partially upward from the wound.
